(19) United States
(12) Patent Application Publication
(10) Pub. No.: US 2003/0064110 A1
(43) Pub. Date: Apr. 3, 2003

(54) Method and composition for treating aphthous stomatitis

(76) Inventor: Terence M. Dolak, Andover, NJ (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/151,445

(22) Filed: May 20, 2002

Related U.S. Application Data
(63) Continuation-in-part of application No. 09/952,119, filed on Sep. 14, 2001, now abandoned.

(51) Int. Cl. A61K 35/14; A61K 35/64

(52) U.S. Cl. 424/539

(57) ABSTRACT

The invention provides a method and composition for treatment of Aphthous Stomatitis by applying thereto an effective amount of a composition comprising from about 0.5 to 25%, preferably about 0.5 to 10%, by weight of propolis extract in a suitable vehicle for topical administration to the oral mucosa. The compositions preferably contain a protectant, an emulsifier and suitable flavoring agents. The subject compositions possess enhanced activity in the treatment of aphthous ulcers in that they stop the progression at the stage when they are applied and promote full healing, generally within 36 to 48 hours.

### RELATED APPLICATIONS

This application is a continuation-in-part of copending U.S. patent application Ser. No. 09/952,119, filed Sep. 14, 2001.

### FIELD OF THE INVENTION

This invention relates to a method and composition for the treatment of idiopathic aphthous ulcers or canker sores.

### BACKGROUND OF THE INVENTION

Aphthous Stomatitis, commonly known as canker sores, are painful ulcers on the movable oral mucosa, occurring singly or in groups. They are generally classified as minor ulcers, the most common form, which are less than 1 cm in diameter, last 10-14 days and typically heal without scarring, and major ulcers, typically greater than 1 cm in diameter, lasting weeks to months and often healing with scarring. Recurrent attacks of aphthous ulcers are common, with two or three ulcers occurring during each attack, however ten to fifteen ulcers are not uncommon in some individuals. Women are affected more often than men. Localization of the ulcers is helpful in differentiating this recurrent condition from recurrent Herpes simplex lesions in the oral cavity. Involvement of the nonkeratinizing mucosa, for example, the labial mucosa, floor of the mouth, ventral tongue, buccal mucosa and soft palate, is generally characteristic of recurrent aphthous ulcers. On the other hand, ulcers on the hard palate, dorsal tongue, attached gingivae, or lips suggest a Herpes infection.

The etiology or pathogenesis of canker sores is unknown. Several factors point toward a localized immune reaction. Impaired cellular immunity has been implicated as a cause of larger recalcitrant ulcers. Deficiencies of iron, vitamin B₁₂, and folic acid are known to increase susceptibility. Stress and local trauma are usually the predominant precipitating factors, Wolverson, Recurrent Aphthous Ulcers, Comprehensive Dermatologic Drug Therapy, pp 773-774, W. B. Saunders Company, Philadelphia, 2001.

The topical treatment of aphthous stomatitis is generally focused on relieving symptoms. For example, the use of a topical anesthetic such as lidocaine (viscous) or benzocaine, as an oral rinse within about three hours before a meal will provide short term relief by minimizing the discomfort caused by eating. A dental protective paste, such as Orabase®, applied four times a day will generally prevent or minimize irritation of the ulcers by the teeth, dental appliances and oral fluids. An application of triamcinolone acetonide in an emollient dental paste will reduce discomfort and promote healing.

In addition, a coating agent such as sucralfate may be helpful. Sucralfate is available as a 10% suspension (Carafate® suspension) which can be held temporarily in the mouth to achieve its coating action. Sucrafate is primarily utilized to enhance healing of duodenal ulcer healing, due to the formation of an ulcer-adhering complex that covers and protects the ulcer. The drug is advantageous in that minimal gastrointestinal absorption occurs. Finally, Amlexanox (Aphthasol® 5% topical paste) has been shown to be an effective topical treatment for aphthous ulcers. Amelexanox is the only therapeutic agent approved by the Food & Drug Administration for the treatment of aphthous stomatitis and is available only on prescription.

Several therapeutic agents have been utilized to treat aphthous ulcers systemically. For multiple lesions, tetracycline oral suspension at a dosage of 250 mg four times a day over a ten day course of therapy has been shown to be effective. The suspension, which is held in the mouth for 2-5 minutes before swallowing to coat ulcers, generally brings symptomatic relief within a day. However, treatment must be repeated for each new attack. For severe episodes, corticosteroid therapy is indicated. Other potent pharmaceutical agents that have been reported in the literature as having utility in the treatment of aphthous stomatitis include the vasodilator pentoxifylline (Trental®) and colchicine, the latter typically being used in combination with topical and systemic corticosteroids. These are powerful drugs and their use must take into consideration both potential adverse effects and contraindications. Off-label use of thalidomide (Thalomid®) has been reported in the literature to have achieved a cure rate almost ten times that for control patients. However, given the long history of the drug with FDA and its side effects and contraindications, it is available only under a special monitored program.

It is clear from the foregoing discussion that there is a need for a preparation effective for the treatment of aphthous stomatitis that is safe and can be obtained without a prescription. Such preparations are provided in accordance with the present invention by the discovery that propolis is an effective agent in the treatment of aphthous stomatitis. Propolis has been used in both water-based and oil-based preparations to treat viral outbreaks. However, to the inventor's knowledge, propolis has not been taught as an effective treatment for Aphthous Stomatitis.

Busciglio, U.S. Pat. No. 4,748,022, discloses compositions for the treatment of pain and inflammation associated with lesions, such as, Herpes simplex, Herpes labilis, Herpes progenitalis, chicken pox lesions, Herpes genitalis . . . and recurrent aphthous ulcers. The disclosed compositions are composed of five components: diphenhydramine, lidocaine, aloe vera gel, propolis and sufficient base to raise the pH to 8-9. Propolis is included in the disclosed compositions because “applications of bee propolis are reported to be the promotion of healing, relief of pain, antibiotic action, among others.” The results in a comparative test showed that a test composition that did not contain the propolis and the aloe vera gel required an average of 7.1 days to heal test lesions, a composition containing all five ingredients required an average of 4.8 days to heal and a third composition that omitted only the propolis required only 3 days. In each instance, the treated ulcers included aphthous ulcers. Although one patient using the third composition stated that it did not heal the lesions, the results overall do not support the efficacy of propolis since the composition without it was over 30% more efficacious than the complete composition. Contrary to expectations, the addition of propolis to the third composition decreased the healing efficacy of the other ingredients, the beneficial effect of aloe vera gel in particular.

In contrast to the foregoing inconclusive teachings concerning propolis, it has been found in accordance with the present invention that propolis in a suitable vehicle provides a safe, effective treatment for Aphthous Stomatitis.

### SUMMARY OF THE INVENTION

The present invention relates to topical preparations and methods for the treatment of Aphthous Stomatitis that reduce the healing time and stops the development of ulcers on contact with full healing, generally in 36 to 48 hours. In particular, the present invention provides a composition for the treatment of Aphthous Stomatitis comprising: propolis extract in a suitable vehicle suitable for topical application to the oral mucosa. The present invention also provides a method for the treatment of idiopathic aphthous ulcers comprising applying to the ulcer an effective amount of a topical composition containing propolis extract.

### DETAILED DESCRIPTION OF THE INVENTION

The present invention provides topical compositions and method for the treatment of Aphthous Stomatitis. Although canker sores as they are commonly know do not follow the well defined stages of lesions that are of viral origin, they are often characterized by early pain or trauma before the lesion begins to develop. The difference in the pattern of development is but further evidence supporting the premise that canker sores are not viral and are not contagious like viral lesions. In the early stage of development, aphthous ulcers have a yellow or white slough and a typically well-defined marginal erythema. The progression of the ulcers over the period of their existence is typically through ulceration, crust forming and healing.

The great majority of patients afflicted with canker sores have the minor type as described above. It is not known with certainty what causes canker sores, and what causes some individuals to have infrequent minor canker sores lasting only a short period, whereas others are afflicted with the major type that can last up to eight weeks of more. In any event, Aphthous Stomatitis is a source of incessant irritation and discomfort to the patient, particularly with ulcers of the major type where the pain and discomfort can be severe. For brevity, the term “ulcer” as utilized herein shall refer to any and all stages of the development of a canker sore, whether or not such stage can be clearly defined. In accordance with the present invention, application of the subject compositions not only dramatically reduces the healing time of Aphthous Stomatitis, but also stops the normal progression of development of the ulcer from its stage of progression at the time of the initial application. On the average, the present compositions reduce healing time of Aphthous Stomatitis to 36 to 48 hours.

The compositions of the present invention are those recognized in the pharmaceutical arts as being suitable for topical application to the oral mucosa and include, without intended limitation, creams, lotions, liquid emulsions, gels and the like. The present compositions comprise from about 0.5 to 25%, preferably from about 0.5to 10%, by weight of propolis extract with a suitable vehicle. The subject compositions preferably additionally comprise from about 1 to 50%, by weight of a suitable protectant and from about 1% to about 20% by weight of a suitable emulsifier.

The protectant forms a barrier over the lesion to help protect against irritation due to trauma from contact with teeth, the tongue and abrasive surfaces on certain foods. The protectant also aids in protecting the ulcers against irritation causes by spicy and/or hot foods, and acts to retard the loss of the active ingredient to the action of saliva. The protectant comprises at least one member selected from the group consisting of allantoin, aluminum hydroxide gel, cocoa butter, dimethicone, glycerin, kaolin, pyridoxine hydrochloride, topical starch, petrolatum, and white petrolatum. Preferred protectants include aluminum hydroxide gel, kaolin and topical starch for forming a physical barrier and cocoa butter, dimethicone, petrolatum and white petrolatum for providing a hydrophobic environment at the site of application. Preferred concentrations of the protectant in the subject compositions will vary within the broad range given above depending on the specific protectant. For example, dimethicone will be utilized in from about 1 to 2% by weight, whereas petrolatum will be utilized in from about 20 to 50% by weight.

The emulsifier of the present composition provides a means of achieving a molecular dispersion of the active principals in propolis extract, the majority of which have limited water solubility. The poor solubility impedes the penetration of the active principals, particularly the flavonoid components and also limits the amount of propolis extract present in the subject compositions. The emulsifier comprises at least one member selected from the group consisting of sorbitan derivatives, particularly sorbitan esters with fatty acids such as oleic acid, alkoxylated alcohols, polymeric ethers, glycerol esters, poly(oxyethylene-oxypropylene)-methylpolysiloxane copolymers and their derivatives and water soluble salts of fatty acids with ammonia, alkanolamines, low molecular weight amines and alkali metals, such as sodium and potassium. Preferred emulsifiers include one or more of polysorbate 60, Polysorbate 80 and other sorbitan ester emulsifiers.

In addition to the preferred ingredients discussed above, the compositions of the present invention may contain other ingredients such as are recognized by those skilled in the pharmaceutical compound arts as being typically present in such formulations. These include, without intended limitation, one or more preservatives, osmotic regulators, flavors, colorants and the like. It will be appreciated that the compounding of the compositions of the present invention will be carried out utilizing some or all of these ingredients depending of the type of preparation desired. Regardless of the form of the compositions of the present invention, it will generally be necessary to add flavors to mask the taste of the propolis extract.

In addition to the choice of these additional ingredients, the intended form of the composition will influence the choice of the certain of the essential ingredients as well, e.g. ingredients that are liquid or semisolid will be utilized to prepare a lotion and those of a higher molecular weight will be used to prepare creams, gels and the like. The choice of such ingredients is considered to be within the purview of the person skilled in the art of pharmaceutical compounding for a given type of preparation. Where the subject compositions are in a fluid form, such as a solution, dispersion, emulsion or other similar preparation, the vehicle can be water, a hydroalcoholic liquid or other suitable liquid recognized as being suitable for compositions that are intended for use in the oral cavity. The subject compositions may be simple compositions comprising only propolis extract and a fluid vehicle with suitable agents to mask the flavor thereof. It will be appreciated that the choice of a vehicle is dependent of the solubilities of the ingredients of the compositions and the desired final consistency. The present compositions are preferably formulated to be hypoallergenic and are packaged in an antiseptic condition to minimize the possibility of complicating infections.

It has been found in accordance with the present invention, that application of the subject compositions at an early stage of the development of a canker sore will substantially retard its development as described above and promote complete healing, typically in 36 to 48 hours.

The present invention is further illustrated by the following examples that are not intended in any way to be limiting thereon.

### EXAMPLE I

A cream containing 4% by weight of propolis extract was prepared from Formula 1 given below.

Formula 1

| Ingredient | Quantity | | Ingredient | Quantity | |
|---|---|---|---|---|---|
| Propolis Extract | 4 | g | Cetyl Alcohol | 2 | g |
| Ethanol | 4 | g | Glyceryl Stearate | 1 | g |
| Dimethicone | 2 | g | Propylene Glycol | 5 | g |
| Stearic Acid | 7 | g | Tocopherol Acetate | 0.2 | g |
| Polysorbate 80 | 3 | g | Methyl Paraben | 0.3 | g |
| PEG-100 Stearate | 1 | g | Propyl Paraben | 0.1 | g |
| Beeswax | 0.5 | g | Triethanolamine | 1 | g |
| Carbomer | 0.5 | g | Disodium EDTA | 0.1 | g |
| Hydrogenated | 3 | g | Water | q.s. 100 | g |
| Polyisobutene | | | | | |

The dimethicone, PEG-100 stearate, beeswax, hydrogenated polyisobutene, cetyl alcohol, glyceryl stearate, tocopherol acetate, methyl and propyl paraben were combined in a suitable vessel with heating to 70-80° C. The resulting anhydrous mixture was held at 70-80° C. with continuous mixing until uniform.

In a second vessel, stearic acid, carbomer, triethanolamine, disodium EDTA and 60 mL of water were combined and gradually heated to 70-80° C. until the resulting aqueous mixture became uniform. The aqueous mixture was slowly added to the anhydrous mixture at 70-80° C. with very rapid mixing. The resulting mixture was homogenized to form an emulsion that was subsequently cooled to 40-50° C.

The propolis extract was placed in a third vessel with the ethanol and propylene glycol gradually added thereto with stirring, followed by the polysorbate 80. The temperature was gradually increased to 40° C. with continuous mixing. When the mixture became uniform, it was slowly added to the homogenized emulsion with rapid stirring. The emulsion was then brought to final weight by the slow addition of the required amount of water with continued blending. The emulsion was again homogenized and cooled to a smooth, creamy consistency.

### EXAMPLE II

Treatment of Aphthous Ulcers

The cream prepared in Example I was tested for its effectiveness against aphthous ulcers in the following three case studies:

### Case I

History of Aphthous Ulcers

A 16 year old male with a history of occasional oral canker sores (idiopathic aphthous ulcers) approximately one every two months. His symptoms included sharp pain centered on the canker sores and infection. His blisters ranged from 1 mm to 5 mm and typically remained for 7-10 days.

Composition Treatment and Results

The patient reported almost immediate relief from pain upon application of the composition to a 3 mm canker sore located on the inside of his lower lip. The composition was reapplied 4 times over a 12 hour period. He indicated that the canker sore was almost gone by the next morning and that he did not need to utilize the composition thereafter. All visible signs of canker sores disappeared by the third day.

### Case II

History of Aphthous Ulcers

A 47 year old male with a history of occasional oral canker sores (idiopathic aphthous ulcers) approximately one every three months. His symptoms included sharp pain centered on the canker sores and infection. The sores formed after a physical break in the lining of the mouth. His blisters ranged from 1 mm to 5 mm and typically remained for 7-10 days.

Composition Treatment and Results

The patient developed a canker sore on the inside of his lower lip as a result of his tooth piercing his lower lip. The composition was not applied until the area was swollen, inflamed and very painful. He reported almost immediate relief from pain upon application of the composition to a 3 mm canker sore located on the inside of the lower lip. The composition was reapplied every 3-4 hours over the next 3 days. He indicated that inflammation, redness and pain were significantly reduced during this time. The canker sore was completely healed by the end of the third day.

### Case III

History of Aphthous Ulcers

A 12 year old male with a history of occasional oral canker sores (aphthous ulcers) approximately one every three months. His symptoms included sharp pain centered on the canker sores and infection. His blisters ranged from 1 mm to 5 mm and typically remained for 7-10 days.

Composition Treatment and Results

The patient applied the composition to a 2-3 mm canker sore on the inside of his cheek every 3-4 hours for two days. He indicated that he obtained almost immediate relief from pain and felt little or no discomfort during this time. All visible signs of the canker sores disappeared by the third day.

### EXAMPLE III

A solution containing 10% by weight of propolis extract was prepared by heating 90 grams of propylene glycol to 45° C. and adding 10 grams of propolis extract with continuous mixing. The mixture was held at 45° C. with continuous mixing until all of the propolis extract dissolved. The mixture cooled to a slightly viscous solution.

### EXAMPLE IV

A solution containing 10% by weight of propolis extract was prepared by combining 20 grams of propolis extract, as a 50% tincture in ethanol, with 80 grams of propylene glycol with stirring at room temperature. The mixture was stirred until a uniform solution was obtained.

### EXAMPLE V

A solution containing 5% by weight of propolis extract was prepared from formula 2.

Formula 2

| Ingredient | Quantity | | Ingredient | Quantity | |
|---|---|---|---|---|---|
| Propolis extract | 5 | g | Propylene glycol | 10 | g |
| Ethanol | 5 | g | Tocopherol Acetate | 0.2 | g |
| Dimethicone | 2 | g | Polyaminopropyl | 4 | g |
| Polysorbate 60 | 6 | g | Biguanide (20% solution) | | |
| PEG-100 Stearate | 2 | g | Water | q.s. 100 | g |

The propolis extract, ethanol, dimethicone, PEG-100 stearate, tocopherol acetate, and propylene glycol were combined in a suitable vessel with heating to 70-80° C. The resulting anhydrous mixture was held at 70-80° C. with continuous mixing until uniform.

In a second vessel, polysorbate 60, the polyaminopropyl biguanide solution and 60 mL of water were combined and gradually heated to 70-80° C. until the resulting aqueous mixture became uniform. The aqueous mixture was slowly added to the anhydrous mixture at 70-80° C. with very rapid mixing. The resulting emulsion was then brought to final weight by the slow addition of the required amount of water with continued blending. The emulsion was homogenized and cooled to a smooth, creamy consistency.

Limited testing has shown the formulae in Examples III-V to be as effective as the formula prepared in Example I, but more convenient to use. Each formulation tested contained a flavoring agent to mask the taste of the propolis extract.

What is claimed is:

1. A composition for the treatment of Aphthous Stomatitis comprising from about 0.5 to 25% by weight of propolis extract in a suitable vehicle for topical administration to the oral mucosa.

2. A composition in accordance with claim 1, wherein the composition is an emulsion.

3. A composition in accordance with claim 1, wherein the composition is a gel.

4. A composition in accordance with claim 1, wherein the composition is a solution.

5. A composition in accordance with claim 1, wherein said composition contains from about 0.5 to 10% by weight of propolis extract.

6. A composition in accordance with claim 1, wherein said composition additionally contains a protectant comprising at least one member selected from the group consisting of allantoin, aluminum hydroxide gel, cocoa butter, dimethicone, glycerine, kaolin, pyridoxine hydrochloride, topical starch, trolamine, petrolatum, and white petrolatum.

7. A composition in accordance with claim 1, wherein said composition is an emulsion, said composition additionally containing an emulsifier comprising at least one member selected from the group consisting of sorbitan derivatives, alkoxylated alcohols, glycerol esters, poly(oxyethylene-oxypropylene)-methylpolysiloxane copolymers, and water soluble salts of fatty acids with ammonia, alkali metals, low molecular weight amines and alkanolamines.

8. A composition in accordance with claim 7, wherein the emulsifier comprises one or more members selected from the group consisting of Polysorbate 80, Polysorbate 60 and other sorbitan ester emulsifiers.

9. A composition in accordance with claim 1, additionally containing a protectant comprising at least one member selected from the group consisting of allantoin, aluminum hydroxide gel, cocoa butter, dimethicone, glycerin, kaolin, pyridoxine hydrochloride, topical starch, petrolatum, and white petrolatum.

10. A composition in accordance with claim 9, wherein said protectant is selected from the group consisting of aluminum hydroxide gel, kaolin, topical starch, cocoa butter, dimethicone, petrolatum and white petrolatum.

11. A method for treatment of lesions of Aphthous Stomatitis comprising thereto an effective amount of a composition comprising from about 0.5 to 25% by weight of propolis extract in a suitable vehicle for topical administration to the oral mucosa

12. A method in accordance with claim 11, wherein said composition contains from about 0.5 to 10% by weight of propolis extract.

13. A method in accordance with claim 11, wherein said composition additionally contains a protectant comprising at least one member selected from the group consisting of allantoin, aluminum hydroxide gel, cocoa butter, dimethicone, glycerine, kaolin, pyridoxine hydrochloride, topical starch, trolamine, petrolatum, and white petrolatum.

14. A method in accordance with claim 11, wherein said composition is an emulsion, said composition additionally containing an emulsifier comprising at least one member selected from the group consisting of sorbitan derivatives, alkoxylated alcohols, glycerol esters, poly(oxyethylene-oxypropylene)-methylpolysiloxane copolymers, and water soluble salts of fatty acids with ammonia, alkali metals, low molecular weight amines and alkanolamines.

15. A method in accordance with claim 14, wherein the emulsifier comprises one or more members selected from the group consisting of Polysorbate 80, Polysorbate 60 and other sorbitan ester emulsifiers.

16. A method in accordance with claim 11, wherein said composition additionally contains a protectant comprising at least one member selected from the group consisting of allantoin, aluminum hydroxide gel, cocoa butter, dimethicone, glycerin, kaolin, pyridoxine hydrochloride, topical starch, petrolatum, and white petrolatum.

17. A method in accordance with claim 11, wherein said protectant is selected from the group consisting of aluminum hydroxide gel, kaolin, topical starch, cocoa butter, dimethicone, petrolatum and white petrolatum.

18. A method in accordance with claim 11, wherein the composition is in the form of an emulsion.

19. A method in accordance with claim 11, wherein the composition is in the form of a gel.

20. A method in accordance with claim 11, wherein the composition is in the form of a solution.
